(19) United States
(12) Patent Application Publication
(10) Pub. No.: US 2004/0197377 A1
(43) Pub. Date: Oct. 7, 2004

(54) 5-HTP composition

(76) Inventor: Marshall Anlauf Thompson, Camarillo, CA (US)

Correspondence Address: Marshall A. Thompson, 1253 N. Modesto, Camarillo, CA, 93010, US

(21) Appl. No.: 10/407,782

(22) Filed: Apr. 3, 2003

(51) Int. Cl. A61K 47/00; A61K 31/405

(52) U.S. Cl. 424/439; 514/419

(57) ABSTRACT

A prepackaged liquid Composition that is intended for oral consumption and includes the ingredient, 5-Hydroxytryptophan (5-HTP) in an amount of between 4.0% and 0.0001% of the final consumable composition.

### BACKGROUND

Knowledge of 5-Hydroxytryptophan (5-HTP) is not new, in fact testing has been performed for decades. As a natural extract of the seed of the Griffonia plant many people have been attracted by test findings that suggest 5-HTP can perform as well or even out perform synthetic drugs for a multitude of health conditions.

While 5-HTP may not be able to cure catastrophic diseases such as cancer. It's unique ability to positively address many negative health events, conditions or symptoms could potentially make it one of the most significant health products available to the public. It is the intent of this concept to be able bring this potential to the general public in a form that will be accepted and utilized.

Serotonin

Serotonin also know as 5-Hydroxytryptamine (5-HT) is derived from the amino acid Tryptophan. It occurs in the brain and intestinal tissue, platelets, and certain connective tissue cells. A strong stimulator of blood-vessel construction and a neurotransmitter, Serotonin concentrates in certain brain areas, especially the mid-brain and hypothalamus.

No other physiological substance known possesses such diverse actions in the body and brain as Serotonin. That is why changes in Serotonin levels can cause such basic changes in a persons well being.

The effects of Serotonin are so basic in fact that if an individual feels poorly no matter the cause or reason their Serotonin levels are low. On the opposite side as levels rise the individual will feel more positive.

Introduction

5-Hydroxytryptophan (5-HTP) is an amino acid that is extracted from the seeds of the West African Griffonia plant. Intake of 5-HTP has been shown to be effective in aiding in production of the neurotransmitter “Serotonin”. More effective in fact than drugs already on the market that are intended to produce Serotonin. This is a result of 5-HTP having a closer chemical make-up to Serotonin than the existing drugs which make it easier for the body to convert it into Serotonin.

As to the reason why an individual would desire to increase their Serotonin levels, we only need look at the some of the conditions related to low Serotonin levels. Depression, Anxiety, Food Cravings, Drug dependence, insomnia, to name a few.

5-HTP represents tremendous potential health benefits. However to be effective it must first be used and to be used it must be delivered in a manor desirable to the public.

Beverages appear to be the method most in favor at this time. For example there are drinks with claims of energy, vitamins, weight loss, weight gain, fiber, stop smoking, and on and on. Sales statistics for these prepackaged health beverages verify there immense popularity.

An obstacle to overcome with regard to creating a 5-HTP liquid Composition is that it is a much more difficult task to dilute 5-HTP in water than most other nutritional ingredients. This hurdle has now been overcome, thereby making this 5-HTP liquid composition a real product and not just a concept.

Given the huge spectrum of conditions effected by Serotonin. A liquid composition that increased Serotonin levels would offer potential positive health benefits far beyond the aspirations of even the most ambitious claims of other health drinks, and there is no more effective mechanism to increase Serotonin levels than 5-HTP.

No matter how great the potential of any concept, unless it can be presented to the public in a form that is useful and appealing, all the benefits the could be enjoying will remain only potential and not the fulfillment of the concepts intent.

At this point in time the all the tremendous health benefits 5-HTP could be offering to the public are being horrendously under utilized.

While it is true that the first initial step has occurred and 5-HTP has been processed into pill form. It is also true that this step is years old and still despite all the potential good 5-HTP could bring to the public, it languishes in obscurity. All because the current delivery system is unappealing which is causing 5-HTP to be under utilized.

Examples of how a liquid can alter the convenience, desirability and utility of an existing substance is can be found in both Westman (U.S. Pat. No. 6,211,194) and Thompson (U.S. Pat. No. 6,268,386). In both a liquid was selected as a delivery system for another naturally occurring substance (Nicotine) that was derived from a plant (Tobacco). In both cases the liquid delivery system was found to substantially alter the utility from other existing Nicotine delivery systems. The same holds as true for 5-HTP. A prepackaged liquid composition will allow 5-HTP to be presented in a more appealing and useful manor and will greatly enhance the prospects for 5-HTP to be at last utilized by a much larger segment of the general population.

However while sales statistics strongly show the appeal beverages have with the general public. What the public can not decide upon is which beverage vessel or type of beverage is desired. It would be a shame if after all the effort to bring the benefits of 5-HTP to an individual they chose not to drink it because it was an unappealing in it presentation.

In accordance with numerous Food And Drug Administration (FDA) label guidelines, ingredients that make up a beverage are listed by amount, with the primary ingredient listed first. These most often are Water, Carbonated Water and Natural Juices (fruit and vegetable). Since the FDA recognizes these as separate ingredients with unique properties, so they shall be treated here.

Water is also the primary ingredient in the bulk of Alcoholic beverages with Alcohol not becoming the primary ingredient until a listing of 100 proof.

Other beverage segments that could contain 5-HTP include but are not limited to Favored Water, Soda, Functional beverages, Energy drinks, Diet drinks, Weight loss beverages and Shakes. While all these beverage variations appeal to a specific public segment all share one of the 3 listed primary ingredients. Likewise flavoring of one of the 3 primary ingredients is very important when trying to appeal to the greatest number of people possible.

Over many years existing beverage manufactures have discovered the vessel containing the liquid needed to be more task specific. Originally there was the glass beverage bottle, then in a effort to broaden the appeal beverage cans appeared, then plastic bottles. More recently beverage boxes, packets, pouches, new bottle and can designs and materials have appeared. With each appealing to a selected sub segment of the market.

For convenience a small vessel such as a packet or vial may contain a concentrated 5-HTP liquid composition that could be added to larger amount of liquid.

At the other extreme a 5-HTP liquid composition concentrate could be in bulk contains, to be mixed in a ratio with a liquid at a later time.

### FIELD OF INVENTION

Depression, Anxiety, Insomnia, Overeating, Headaches, Mood swings, Fibromyalgia, Drug and Alcohol dependence are of some of perhaps many conditions that may all just be basically different manifestations of the same underlying disorder. That being low levels of Serotonin in a persons brain. The most obvious solution is that if low levels of a chemical are the cause of negative health events then intake of additional amounts of that substance would correct the problem. In the case of Serotonin that option is not available because while Serotonin is a desired neurotransmitter in the brain, an individuals body possess chemical barriers that prevent Serotonin from moving freely throughout the body to the desired destination.

Therefore what is needed is a substance that can cross these barriers and then be converted directly into Serotonin, what a chemist would refer to as a direct precursor and 5-HTP is best precursor not only available but possible.

5-HTP Compound and Insomnia

Decreasing Serotonin levels in the brain disrupt normal sleep patterns. Whereas increased levels of Serotonin generally promote sleep.

5-HTP is able to promote sleep with out the drugged or hangover feelings associated with powerful sedatives.

Melatonin also helps promote sleep and some of the increased Serotonin produced by the body as a result of ingesting 5-HTP is converted directly into melatonin. Thus at least of the sleep inducing effects of 5-HTP is a result of increased melatonin levels.

5-HTP Compound and Depression

The concept that Serotonin was a crucial neurotransmitter related to depression was first proposed in the mid 1970's. That conclusion being based at least partially upon the observation that many individuals diagnosed with depression had low levels of a metabolite of Serotonin called (5-HIAA) in the fluids that surrounds the brain and spinal cord. Low 5-HIAA suggests that the brain is not producing and there by not metabolizing a normal amount of Serotonin.

Therefore if low Serotonin level are linked to the on set of depression, does restoring this deficit bring a person back to “normal”. The answer to that based on overwhelming evidence suggests that it does.

To restore this normal of Serotonin virtually all existing anti-depressant remedies use a method of slowing down the degradation of Serotonin already produced. 5-HTP however takes the opposite approach by increasing the amount of Serotonin nerve cells produced.

5-HTP Compound and Anxiety

A normal level of uncertainty or anxiety is normal and can actually aid performance in many situation. Excessive anxiety can be a debilitating disorder and while anxiety and depression are related, the trigger mechanisms are more complex for anxiety. Also anxiety can be divided into two categories short term “panic attacks” and long term anxiety such as “obsessive compulsive disorder” (OCD) or “post traumatic stress disorder” (PTSD).

Panic attacks are caused by a rapid depletion of tryptophan, leading to a reduction in Serotonin levels. 5-HTP increases the production of Serotonin thereby reducing the severity of these attacks.

Long term anxiety is a result of Serotonin receptors that have become hypersensitive. With continued stimulation by increase Serotonin, over time these receptors eventually become less sensitive, this is know as “downregulation”, and anxiety levels return to normal.

5-HTP Compound and Headaches

There are two types of headaches, vascular and non-vascular. Vascular headaches result from the dilation of blood vessels in the head. The pounding pain is the result of blood pumping through these vessels with each beat of the heart. Migraines are a form of vascular headaches. The good news is that Serotonin acts to constrict blood vessels and relieve pain. 5-HTP's effects on Serotonin thus makes it a valuable asset in preventing these types of headaches.

For non-vascular as well as vascular headaches, it has been found that the more Serotonin present, the less vulnerable a person is to pain. This is due to the fact that 5-HTP increase the levels of endorphins. Endorphins are chemically related to morphine are the bodies natural pain killers. Mounting evidence suggests that 5-HTP can boost endorphin levels by increasing Serotonin which regulates the production and release of endorphins.

5-HTP Compound and Sleep Apnea

Sleep Apnea to put it simply is trouble breathing while sleeping and this is caused by low levels of Serotonin. The nerves that control breathing require an adequate supply of Serotonin. Also, Serotonin receptors control the release of hormones such as cortisol. One role of cortisol is to help control the muscles needed for breathing.

5-HTP Compound and Fibromyalgia

Fibromyalgia is characterized by chronic muscle aching, multiple tender points, fatigue, morning stiffness and disturbed sleep. Taking 5-HTP can generally improve or reduced these conditions.

5-HTP Compound and Obesity

In many cases obesity is a adverse result of an other condition(s), such as depression or anxiety. In both cases 5-HTP has been show to at the very least reduce the impact of these conditions and thereby address some of the causes of obesity.

There is also a well regarded theory that some people are genetically born to be fat because there body does not produce sufficient Serotonin, resulting in constant cravings for food to meet this deficiency.

5-HTP Compound and Weight Loss, Carbohydrate Craving

Apart from the conditions that cause obesity is the use of 5-HTP in appetite suppression. An effect of low Serotonin levels in the brain is a craving for food or to be more exact carbohydrates. By feeding the craving that exists only in the brain the individual need only consume foods to meet the nutritional need of the body. Without these cravings a reasonable diet has a much better chance of being effective.

5-HTP Compound and Bulimia

Lower Serotonin levels in the brain can trigger some of the clinical features of bulimia nervosa in individual vulnerable to the disorder. It is also possible depression plays a role as well. Moderate dieting is known to lower blood levels of tryptohan, which may alter Serotonin levels in the brain. Therefore, this maybe one way in which dieting can trigger bulimia in individuals.

5-HTP Compound and PMS

Depression, anxiety, mood swings, increased appetite and fatigue are all common symptoms and are all indicators of low Serotonin levels. This occurs due to physical changes during this time that decrease the availability Serotonin. With 5-HTP, Serotonin levels can return to normal leading to the individual feeling normal again.

5-HTP Compound and Narcolepsy

There is no cure for narcolepsy however the symptoms of excessive daytime sleepiness, sudden sleep onset and cataplexy are very easy to treat. The preferred approach is the good quality nocturnal sleep that can be provide by 5-HTP, and a regular schedule.

5-HTP Compound and Improved Mood

All the previous passages have shown the negative effects of low Serotonin levels and how a return to normal levels can improve various conditions. However a strength of 5-HTP is that can provide a general feeling of wellness without that drugged feeling and side effects associated with use of other substances.

5-HTP Compound and Cocaine

Cocaine blocks the transport or movement of Serotonin in the brain. This makes people with low Serotonin levels more likely to become quickly a addicted to cocaine then a person with normal levels. It is also the depletion of Serotonin that causes the “high” associated with cocaine usage. After cocaine usage it is once again the depleted Serotonin levels that cause such strong withdraw symptoms and such a strong need to use cocaine again and again.

5-HTP Compound and Marijuana

Marijuana usage is often in response to an individuals state of depression or anxiety. Both are conditions that respond very positively to increased Serotonin levels and as such the root cravings that drive marijuana usage can be satisfied by 5-HTP with out the side effects of marijuana.

5-HTP Compound and Alcohol

A small amount of alcohol can increase Serotonin levels thereby creating an improved mood. However large amounts of alcohol an decrease Serotonin levels with a result of increasing depression.

A small amount of alcohol can also ease anxiety, and again as the amount increases the desired effects decrease.

This is because alcohol competes with Serotonin. Initially the production of Serotonin is increased to counter the alcohol effects but as the amount of alcohol increases it begins to mimic the Serotonin. This results in body being deceived into believing that the proper level of Serotonin exists causing it slows it own production. The result of this being increased anxiety and depression.

Also as alcohol is used more often the initial trigger to increase Serotonin levels requires ever greater amount of alcohol.

5-HTP does not mimic Serotonin it triggers it's production and without the side effects of alcohol which are well documented.

5-HTP Compound and Nicotine

The most sought after trait of nicotine products is the general feeling of well being that they convey. This is accomplished by triggering the production of Serotonin in the brain. However as a trigger for Serotonin production nicotine is not nearly as effective as 5-HTP in this regard. This makes 5-HTP a very strong alternative to nicotine products.

What is claimed is:

1. A prepackaged delivery system containing a liquid composition of greater than 100 ml intended for oral consumption. The composition having a primary ingredient of water and includes the ingredient 5-Hydroxytryptophan (5-HTP) in an amount of between 0.0001% and 4.0% of the final composition intended for oral consumption.

2. A prepackaged delivery system for the oral consumption of a liquid composition that contains the ingredient 5-HTP in an amount of between 0.0001% and 5.0% of the composition. The vessel for delivery of this composition being one of the following:
A. A sealed Packet.
B. A sealed Vial.
C. A sealed Pouch.
D. A sealed Cup.

3. As per claim 1, wherein the primary component of the composition is a Natural Juice.

4. As per claim 1, wherein the primary component of the composition is Carbonated Water.

5. As per claim 1, wherein the intent of the composition is to address the condition of Depression.

6. As per claim 1, wherein the intent of the composition is to address the condition of Obesity.

7. As per claim 1, wherein the intent of the composition is to aid Weight Loss.

8. As per claim 1, wherein the intent of the composition is to address the condition of Anxiety.

9. As per claim 1, wherein the intent of the composition is to address the condition of Headaches.

10. As per claim 1, wherein the intent of the composition is to improve Mood.

11. As per claim 1, wherein the intent of the composition is to address the condition of Insomnia.

12. As per claim 1, wherein the intent of the composition is to address the condition of Sleep Apnea.

13. As per claim 1, wherein the intent of the composition is to address the condition of PMS, (Premenstrual Syndrome).

14. As per claim 1, wherein the intent of the composition is to address the condition of Narcolepsy

15. As per claim 1, wherein the intent of the composition is to address the condition of Carbohydrate Craving.

16. As per claim 1, wherein the intent of the composition is to address the condition of Bulimia.

17. As per claim 1, wherein the intent of the composition is to address the condition of Fibromyalgia.

18. As per claim 1, wherein the intent of the composition is to address the condition of a dependency on Alcohol.

19. As per claim 1, wherein the intent of the composition is to address the condition of a dependency on Marijuana.

20. As per claim 1, wherein the intent of the composition is to address the condition of a dependency on Cocaine.

21. As per claim 1, wherein the intent of the composition is to address the condition of a dependency on Nicotine.

22. As per claim 1, wherein the intent is to increase the production of Serotonin.

23. As per claim 1, wherein the intent is to increase the production of melatonin.

24. As per claim 1, wherein the intent is to increase an individuals endorphin level.
